(19) United States
(12) Patent Application Publication
(10) Pub. No.: US 2023/0363379 A1
(43) Pub. Date: Nov. 16, 2023

(54) SUSTAINED RELEASE BODY AND METHOD OF MANUFACTURING SUSTAINED RELEASE BODY

(71) Applicant: KOJIMA INDUSTRIES CORPORATION, Toyota-shi (JP)

(72) Inventors: Takaaki TANAKA, Toyota-shi (JP); Akihiko KOZUKA, Toyota-shi (JP); Akira TSUJI, Toyota-shi (JP); Satoru KATOH, Toyota-shi (JP); Syouhei KAWAGUCHI, Toyota-shi (JP); Saki SUZUKI, Toyota-shi (JP)

(21) Appl. No.: 18/358,025

(22) Filed: Jul. 25, 2023

Related U.S. Application Data
(63) Continuation of application No. PCT/JP2022/000741, filed on Jan. 12, 2022.

(30) Foreign Application Priority Data
Jan. 26, 2021 (JP) 2021-010702

(51) Int. Cl. A01N 25/10; A61L 9/012; A61L 9/04; A01N 35/06; A01P 1/00; B32B 5/18; B32B 27/36; B32B 27/06

(52) U.S. Cl. CPC A01N 25/10; A61L 9/012; A61L 9/042; A01N 35/06; A01P 1/00; B32B 5/18; B32B 27/36; B32B 27/065; B32B 2266/06; B32B 2266/0214; B32B 2255/102; B32B 2255/26; B32B 2307/748

(57) ABSTRACT

A sustained release body includes: a substrate containing a vaporizable active ingredient, the substrate having a structure for releasing a vaporized gas of the active ingredient; and a vapor deposition polymerization film directly formed on a first surface that is one end surface of both end surfaces of the substrate in a thickness direction of the substrate, the vapor deposition polymerization film being configured to receive the vaporized gas of the active ingredient from the first surface of the substrate and sustainedly release the vaporized gas.

### CROSS-REFERENCE TO RELATED APPLICATION

This application is a continuation of International Application No. PCT/JP2022/000741, filed on Jan. 12, 2022 which claims the benefit of priority of the prior Japanese Patent Application No. 2021-010702, filed on Jan. 26, 2021, the entire contents of which are incorporated herein by reference.

### BACKGROUND OF THE INVENTION

The present disclosure relates to a sustained release body and a method of manufacturing the sustained release body.

In the related art, in order to cause an active ingredient such as an antifungal agent, an antimicrobial agent, or a deodorant to act on a desired object over a long period of time, a sustained release body that vaporizes the active ingredient and gradually releases (sustainedly releases) the vaporized active ingredient to the outside has been proposed. In general, the sustained release body includes an active ingredient layer containing a vaporizable active ingredient on a substrate, and includes a sustained release film on the active ingredient layer. In this sustained release body, the sustained release film sustainedly releases a gas (vaporized gas) of the active ingredient vaporized from the active ingredient layer to the outside. This can cause the active ingredient to act on a desired object over a long period of time.

As such a sustained release body, for example, JP 2017-226188 A discloses a sustained release film in which an active ingredient layer is formed on a surface of a substrate film, and an active ingredient permeation layer is formed on the active ingredient layer. In the sustained release film described in JP 2017-226188 A, the active ingredient permeation layer functions as a sustained release film that sustainedly releases a vaporized gas of an active ingredient generated from the active ingredient layer to the outside.

### SUMMARY OF THE INVENTION

However, in the conventional sustained release body described above, a gap is generated between the active ingredient layer and the sustained release film as the vaporized active ingredient is sustainedly released. That is, as exemplified in FIG. 9, in a conventional sustained release body 100, an active ingredient layer 103 is interposed between a substrate 101 and a sustained release film 102. Therefore, as a vaporized gas 103a of the active ingredient contained in the active ingredient layer 103 is sustainedly released from the sustained release film 102 to the outside, the active ingredient layer 103 continues to decrease, and finally, a gap 104 is generated between the active ingredient layer 103 and the sustained release film 102. In this case, since the sustained release film 102 formed of a vapor deposition polymerization film is a thin film, for example, a damage such as a crack 105 illustrated in FIG. 9 may be easily generated in the sustained release film 102 due to an external force caused by bending, pressing, or the like. As a result, the vaporized gas 103a of the active ingredient is excessively released from a damaged portion of the sustained release film 102, and as a result, it is difficult to continue sustained release of the vaporized gas 103a of the active ingredient over a target long period of time.

In that regard, it is desirable to provide a sustained release body that stable sustained release of an active ingredient can be continued over a target long period of time, and a method of manufacturing the sustained release body.

In some embodiments, a sustained release body includes: a substrate containing a vaporizable active ingredient, the substrate having a structure for releasing a vaporized gas of the active ingredient; and a vapor deposition polymerization film directly formed on a first surface that is one end surface of both end surfaces of the substrate in a thickness direction of the substrate, the vapor deposition polymerization film being configured to receive the vaporized gas of the active ingredient from the first surface of the substrate and sustainedly release the vaporized gas.

In some embodiments, a method of manufacturing a sustained release body includes: forming a sustained release film made of a vapor deposition polymerization film on a first surface that is one end surface of both end surfaces of the substrate in a thickness direction of the substrate; and introducing a vaporizable active ingredient into the substrate from a second surface opposite to the first surface, the second surface being another surface of the both end surfaces of the substrate in the thickness direction.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic cross-sectional view illustrating a configuration example of a sustained release body according to an embodiment of the disclosure;

FIG. 2 is a schematic cross-sectional view illustrating a configuration example of the sustained release body according to the embodiment of the disclosure when the sustained release body is used;

FIG. 3 is a flowchart illustrating an example of a method of manufacturing a sustained release body according to the embodiment of the disclosure;

FIG. 4 is a schematic diagram for specifically describing the method of manufacturing a sustained release body according to the embodiment of the disclosure;

FIG. 5 is a schematic diagram illustrating an example of a film forming apparatus for forming a sustained release film of the sustained release body according to the embodiment of the disclosure;

FIG. 6 is a schematic diagram illustrating an example of an application apparatus for introducing an active ingredient into a substrate of the sustained release body according to the embodiment of the disclosure;

FIG. 7 is a schematic diagram illustrating an example of a pasting apparatus for forming a barrier layer of the sustained release body according to the embodiment of the disclosure;

FIG. 8A is a schematic cross-sectional view illustrating a configuration example of a barrier layer film to be pasted on a sustained release film surface of the sustained release body according to the embodiment of the disclosure;

FIG. 8B is a schematic cross-sectional view illustrating a configuration example of a barrier layer film to be pasted on a substrate surface of the sustained release body according to the embodiment of the disclosure; and

FIG. 9 is a schematic cross-sectional view for describing a problem of a conventional sustained release body.

### DETAILED DESCRIPTION

Hereinafter, a preferred embodiment of a sustained release body according to the disclosure and a method of manufacturing the sustained release body will be described in detail with reference to the attached drawings. Note that the disclosure is not limited by the present embodiment. In addition, it should be noted that the drawings are schematic, and a dimensional relationship among elements, a ratio among the elements, and the like may be different from actual ones. Some portions may have different dimensional relationships and ratios among the drawings. In addition, in the drawings, the same components are denoted by the same reference numerals.

Configuration of Sustained Release Body

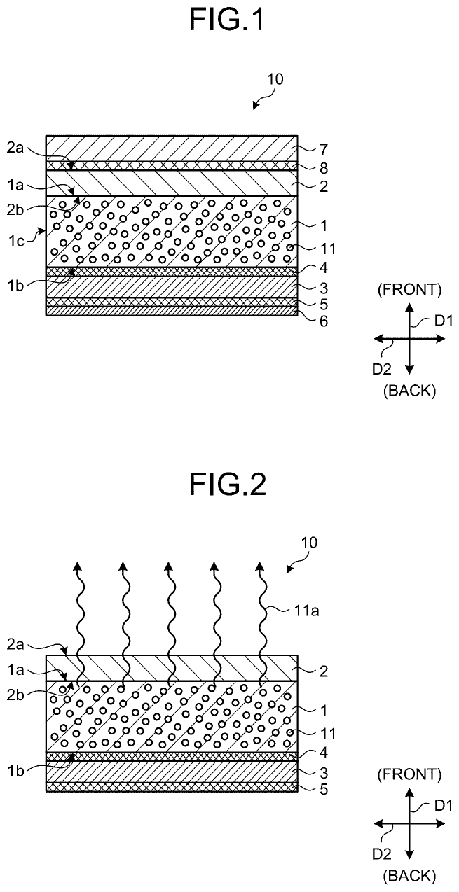

First, a configuration of a sustained release body according to an embodiment of the disclosure will be described. FIG. 1 is a schematic cross-sectional view illustrating a configuration example of the sustained release body according to the embodiment of the disclosure. FIG. 2 is a schematic cross-sectional view illustrating a configuration example of the sustained release body according to the embodiment of the disclosure when the sustained release body is used. FIGS. 1 and 2 illustrate an example of a laminated structure of a sustained release body 10 according to the present embodiment.

Note that, in the present embodiment, for convenience of description, a thickness direction D1 and a width direction D2 of the sustained release body 10 are set, but these directions do not limit the disclosure. One end side (upper side in FIGS. 1 and 2) in the thickness direction D1 is defined as a front side, and the other end side (lower side in FIGS. 1 and 2) in the thickness direction D1 is defined as a back side. The width direction D2 is a direction perpendicular to the thickness direction D1. As for the thickness direction D1 and the width direction D2, the same applies to components of the sustained release body 10.

The sustained release body 10 according to the present embodiment vaporizes an active ingredient to act on a desired object (not illustrated) and sustainedly releases the vaporized active ingredient to the outside. As illustrated in FIG. 1, the sustained release body 10 includes a substrate 1 containing a vaporizable active ingredient 11, a sustained release film 2, barrier layers 3 and 7, adhesive layers 4, 5, and 8, and a protective sheet 6.

As illustrated in FIG. 1, the substrate 1 serves as a support layer supporting the sustained release film 2, and contains the vaporizable active ingredient 11. In addition, the substrate 1 has a structure that releases a vaporized gas 11a (see FIG. 2) of the active ingredient 11 contained therein. Such a substrate 1 is preferably made of, for example, a polymer having a non-crystalline portion or a porous material.

Examples of the porous material constituting the substrate 1 include a porous resin in which many minute pores are formed by subjecting a resin having a solid structure to a treatment such as stretching or perforation, a foamed resin, and a fiber structure. Such a porous material is an advantageous structure from viewpoints of ease of containing the active ingredient 11 and ease of releasing the vaporized gas 11a of the active ingredient 11 because there is a minute space in the porous material and there is a portion connected from the minute space to the outside. In addition, the porous material more preferably has a structure of continuous open cell from a viewpoint that the vaporized gas 11a of the active ingredient 11 is easily released by the continuous open cell in the porous material.

Examples of a resin component constituting the porous resin include a polyester such as polyethylene terephthalate (PET), nylon, polyvinyl chloride, polyethylene, polypropylene, an ethylene-vinyl acetate copolymer, and polytetrafluoroethylene. For example, minute pores in the porous resin can be formed by adding calcium carbonate to polyethylene and stretching the mixture. Examples of the foamed resin include a polyolefin foamed body, a polyurethane foamed body, and a rubber-based foamed body. Examples of the fiber structure include paper, a woven fabric, and a nonwoven fabric.

In addition, the above-described polymer having a non-crystalline portion is a polymer having at least a non-crystalline structure. Examples of the polymer having a non-crystalline portion include a non-crystalline polymer, a mixed polymer of crystal and non-crystal, and rubber. Examples of the non-crystalline polymer constituting the substrate 1 include polystyrene and an acrylic resin. Examples of the acrylic resin include a polymethyl methacrylate resin (PMMA). Examples of the mixed polymer of crystal and non-crystal include a resin having a folded crystal structure in which a crystal structure and a non-crystalline structure are mixed. Examples of the resin having the folded crystal structure include polyethylene, polypropylene, and nylon. Such a non-crystalline structure is an advantageous structure from viewpoints of ease of containing the active ingredient 11 and ease of releasing the vaporized gas 11a of the active ingredient 11 because the active ingredient easily enters and easily permeates a gap between molecular chains.

The active ingredient 11 contained in the substrate 1 is selected according to use of the sustained release body 10. Examples of the active ingredient 11 include various ingredients such as an antifungal agent, an antimicrobial agent, an anticorrosive agent, a rust inhibitor, a deodorant, and a perfume. Alternatively, the active ingredient 11 may be a combination of two or more of these ingredients. Examples of a compound constituting such an active ingredient 11 include hinokitiol, citronellal, green leaf aldehyde (trans-2-hexenal), citronellol, and geraniol. Examples of the use of the sustained release body 10 include use in a box for transportation or storage of an object such as vegetables, fruits, or flowers, and use in packaging of the object. In addition to these, examples of the use of the sustained release body 10 include use in an antifungal sheet (for example, a mildew generation preventing sheet) in house cultivation, an antifungal sheet for measures against tinea in an insole of a shoe sole, an antifungal and deodorant sheet in a footwear box, and an antimicrobial sheet.

Note that the thickness of the substrate 1 is set according to the content of the active ingredient 11, a target sustained release period of the vaporized gas 11a, use of the sustained release body 10, a size at the time of use, and the like. For example, the thickness of the substrate 1 is about 10 μm to several mm.

The sustained release film 2 is a film for sustainedly releasing the active ingredient 11 vaporized from the substrate 1 to the outside. Specifically, as illustrated in FIG. 1, the sustained release film 2 is a vapor deposition polymerization film directly formed on a front surface 1a (first surface) out of both end surfaces of the substrate 1 in the thickness direction D1. That is, the sustained release film 2 is formed by vacuum vapor deposition or the like of two or more types of raw materials on the front surface 1a of the substrate 1. Such a sustained release film 2 receives the vaporized gas 11a of the active ingredient 11 contained in the substrate 1 from the front surface 1a of the substrate 1 and sustainedly releases the vaporized gas 11a as illustrated in FIG. 2 in a state where the barrier layer 7 on a front surface 2a of the sustained release film 2 is removed.

In the present embodiment, the vaporized gas 11a is an active ingredient gas vaporized from the active ingredient 11 in the substrate 1 by volatilization, evaporation, or the like, and as illustrated in FIG. 2, the vaporized gas 11a permeates the substrate 1, passes through a back surface 2b of the sustained release film 2 from the front surface 1a of the substrate 1, and flows into the sustained release film 2. Thereafter, the vaporized gas 11a permeates the sustained release film 2 and is sustainedly released to the outside of the sustained release body 10. At this time, since the sustained release film 2 is a vapor deposition polymerization film formed directly on the front surface 1a of the substrate 1, the sustained release film 2 sustainedly releases the vaporized gas 11a and maintains a state of being in close contact with the front surface 1a of the substrate 1. Therefore, even when the vaporized gas 11a continues to be sustainedly released, the vaporized gas 11a does not accumulate and the active ingredient 11 is not present at a close contact interface between the sustained release film 2 and the substrate 1, that is, between the back surface 2b of the sustained release film 2 and the front surface 1a of the substrate 1.

Such a vapor deposition polymerization film constituting the sustained release film 2 can enhance adhesion between the substrate 1 and the sustained release film 2. That is, the front surface 1a of the substrate 1 is an uneven surface having a large number of minute openings through which the vaporized gas 11a of the active ingredient 11 is released. In a case where the sustained release film 2 is made of a vapor deposition polymer, since the sustained release film 2 comes into close contact with the front surface 1a while following unevenness of the front surface 1a, a gap is less likely to be generated between the sustained release film 2 and the substrate 1, and therefore a crack is less likely to be generated in the sustained release film 2. For example, the sustained release film 2 made of a vapor deposition polymerization film is formed by vacuum vapor deposition and polymerization of two or more types of raw material monomers directly on the front surface 1a of the substrate 1.

The sustained release film 2 has a lower gas permeability than the substrate 1 described above. In the disclosure, the gas permeability is defined by a permeation amount of a gas that permeates a region of a unit area of a film or a layer per unit time. The gas permeability has a unit of [g/m²/day]. That is, the sustained release film 2 releases (sustainedly releases) the vaporized gas 11a generated from the active ingredient 11 in the substrate 1 to the outside by reducing the permeation amount of the vaporized gas 11a based on the gas permeability of the substrate 1 to the permeation amount of the vaporized gas 11a based on the gas permeability of the sustained release film 2.

For example, the gas permeability of the substrate 1 can be measured by performing a water vapor permeability test using a measurement sample of the substrate 1. As a result, as an example of the gas permeability of the substrate 1, a water vapor permeation amount of more than 3000 g/m²/day (>> gas permeability of the sustained release film 2) was obtained. In addition, the gas permeability of the sustained release film 2 which is one of the main components of the sustained release body 10 according to the embodiment of the disclosure can be measured by forming the sustained release film 2 on the substrate 1 and then performing a water vapor permeability test similar to that for the substrate 1. As a result, as an example of the gas permeability of the sustained release film 2, a water vapor permeation amount of 467 g/m²/day was obtained. From the above, it could be confirmed that the gas permeability in a case where the sustained release film 2 was formed on the substrate 1 was lower than the gas permeability of only the substrate 1. That is, it is obvious that the sustained release amount of the vaporized gas 11a of the active ingredient 11 from the substrate 1 can be adjusted by the sustained release film 2.

Note that, in the measurement of the gas permeability, as a measurement sample of the sustained release film 2, a film in which a sustained release film (thickness: 4 μm) made of a vapor deposition polymer was formed on a surface of a substrate (thickness: 75 μm) made of a polyethylene porous material was used. As a measurement sample of the substrate 1, a substrate (thickness: 75 μm) made of the polyethylene porous material was used. The water vapor permeation amount was measured under conditions of a temperature of 20 to 40° C. and a relative humidity of 60% RH or 90% RH on the basis of an isobaric method defined in a moisture sensor method (JIS K 7129A).

The thickness of the sustained release film 2 as described above is set in comprehensive consideration of a target sustained release amount of the vaporized gas 11a of the active ingredient 11 (sustained release amount of the vaporized gas 11a sustainably released to the outside from a region of a unit area of the sustained release film 2 per unit time), the type and content of the active ingredient 11, and the like. For example, the thickness of the sustained release film 2 is preferably in a range of 1 μm or more and 900 μm or less. This is because when the thickness of the sustained release film 2 is less than 1 μm, a crack is likely to be generated in the sustained release film 2, and when the thickness of the sustained release film 2 is more than 900 μm, a sustained release property of the sustained release film 2 may be deteriorated.

The barrier layer 3 is for preventing the vaporized gas 11a of the active ingredient 11 from being released from an unintended surface of the sustained release body 10. Specifically, as illustrated in FIG. 1, the barrier layer 3 is made of, for example, a material having a lower gas permeability than the sustained release film 2 (preferably not transmitting a gas), such as a resin or a metal, and is formed on a back surface 1b of the substrate 1. For example, the barrier layer 3 has the adhesive layer 4 on a front surface thereof, and is pasted on the back surface 1b of the substrate 1 by an adhesive force of the adhesive layer 4. In the present embodiment, the back surface 1b of the substrate 1 is a surface (second surface) opposite to the front surface 1a on which the above-described sustained release film 2 is formed out of both end surfaces of the substrate 1 in the thickness direction D1. That is, the front surface 1a of the substrate 1 is a surface from which release (sustained release) of the vaporized gas 11a of the active ingredient 11 is intended. The back surface 1b of the substrate 1 is a surface from which release of the vaporized gas 11a is not intended. The barrier layer 3 blocks the vaporized gas 11a of the active ingredient 11 from the back surface 1b side of the substrate 1. As a result, the barrier layer 3 prevents the vaporized gas 11a of the active ingredient 11 from being released from the back surface 1b of the substrate 1 from which release of the gas is not intended.

Examples of the resin constituting the barrier layer 3 described above include a crystalline resin having a solid structure and a rubber. Examples of the crystalline resin include polyester and nylon. Examples of the rubber include a vulcanized rubber, a silicone rubber, and a urethane rubber.

Note that the area of a side surface 1c (an end surface in the width direction D2) of the substrate 1 is extremely smaller than that of the front surface 1a of the substrate 1. Therefore, even if the vaporized gas 11a of the active ingredient 11 is released from the side surface 1c of the substrate 1, the release amount of the vaporized gas 11a from the side surface 1c is negligibly small as compared with the release amount of the vaporized gas 11a from the front surface 1a of the substrate 1.

The adhesive layer 5 is for fixing the sustained release body 10 to a desired use place. Specifically, as illustrated in FIG. 1, the adhesive layer 5 is formed on a back surface of the barrier layer 3. For example, before use of the sustained release body 10 (before sustained release of the vaporized gas 11a of the active ingredient 11), the protective sheet 6 that protects the adhesive layer 5 is detachably formed on a back surface of the adhesive layer 5. For example, as illustrated in FIGS. 1 and 2, the protective sheet 6 is appropriately peeled off from the back surface of the adhesive layer 5 when the sustained release body 10 is used. As a result, an adhesive surface of the adhesive layer 5 is exposed, and the sustained release body 10 can be pasted on and fixed to a desired use place.

The barrier layer 7 is for preventing the vaporized gas 11a of the active ingredient 11 from being sustainedly released during an unintended period of the sustained release body 10. Specifically, as illustrated in FIG. 1, the barrier layer 7 is made of a similar material to that of the barrier layer 3 described above, and is detachably formed on the front surface 2a of the sustained release film 2. For example, the barrier layer 7 has the adhesive layer 8 on a back surface thereof, and is pasted on the front surface 2a of the sustained release film 2 by an adhesive force of the adhesive layer 8. Note that the barrier layer 7 itself may be made of an adhesive resin or the like, and may be pasted on the front surface 2a of the sustained release film 2 by an adhesive force of the barrier layer 7 itself. Any technique may be used, but the barrier layer 7 is preferably formed such that the adhesive (adhesive layer 8) does not remain on the front surface 2a of the sustained release film 2 after the barrier layer 7 is peeled off from the sustained release film 2. Such a barrier layer 7 covers the front surface 2a of the sustained release film 2, thereby preventing sustained release of the vaporized gas 11a of the active ingredient 11 from the sustained release film 2 in a period during which sustained release of the vaporized gas 11a of the active ingredient 11 is not intended, such as before use of the sustained release body 10.

Meanwhile, for example, as illustrated in FIGS. 1 and 2, the barrier layer 7 is appropriately peeled off from the front surface 2a of the sustained release film 2 when the sustained release body 10 is used. As a result, the front surface 2a (sustained release surface) of the sustained release film 2 is exposed as illustrated in FIG. 2. As a result, the sustained release film 2 can sustainedly release the vaporized gas 11a of the active ingredient 11 vaporized from the substrate 1 toward a desired object (not illustrated) to the outside of the sustained release body 10.

Method of Manufacturing Sustained Release Body

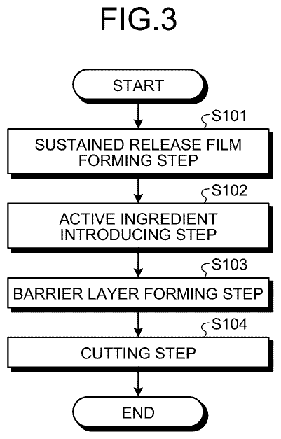

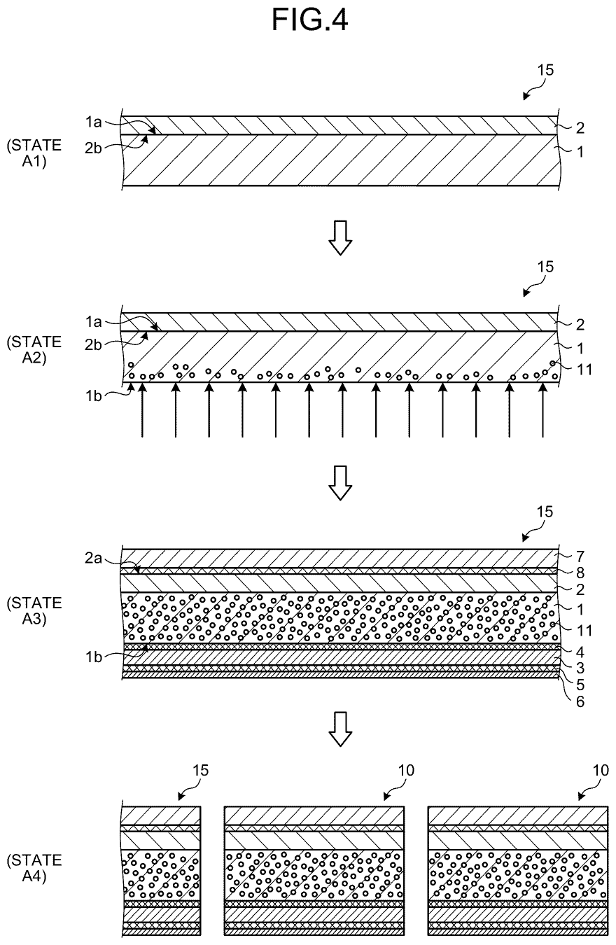

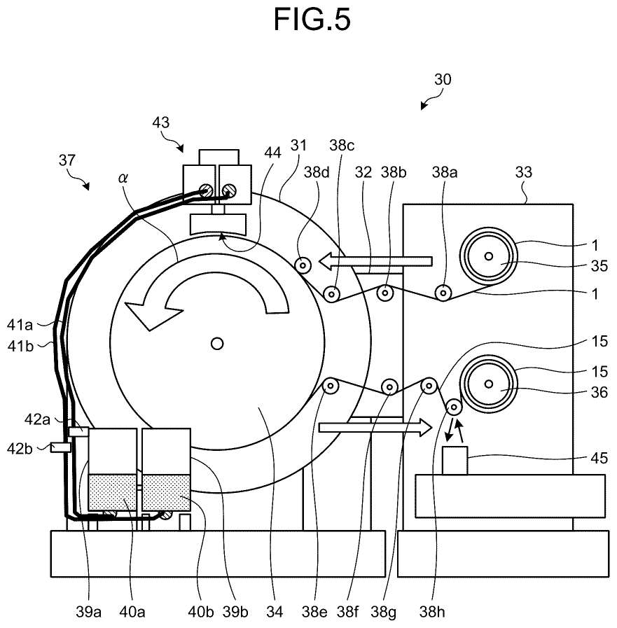

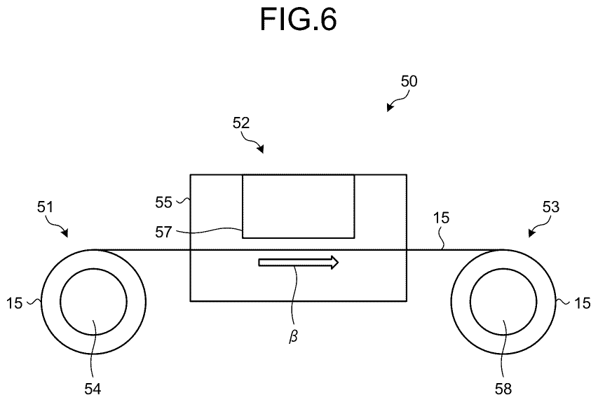

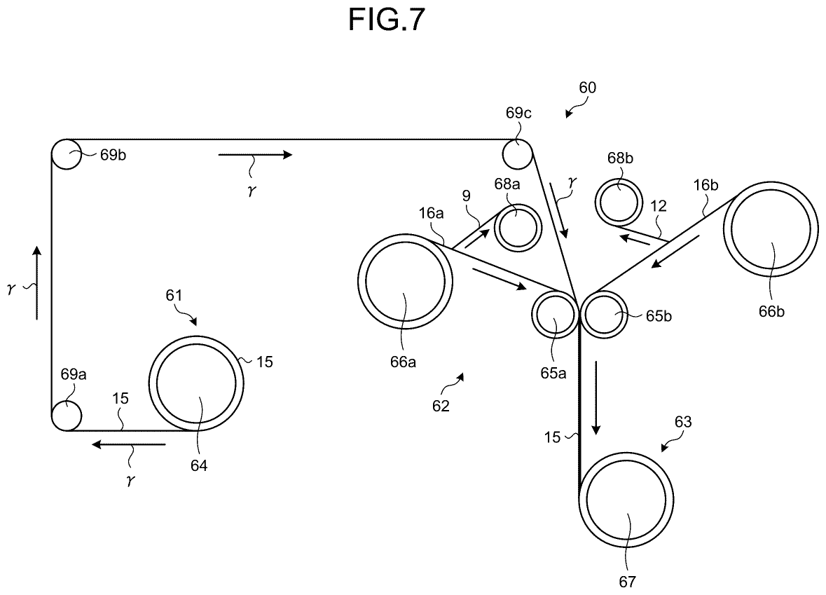

Next, a method of manufacturing the sustained release body 10 according to the embodiment of the disclosure will be described. FIG. 3 is a flowchart illustrating an example of a method of manufacturing a sustained release body according to the embodiment of the disclosure. FIG. 4 is a schematic diagram for specifically describing the method of manufacturing a sustained release body according to the embodiment of the disclosure. FIG. 5 is a schematic diagram illustrating an example of a film forming apparatus for forming a sustained release film of the sustained release body according to the embodiment of the disclosure. FIG. 6 is a schematic diagram illustrating an example of an application apparatus for introducing an active ingredient into a substrate of the sustained release body according to the embodiment of the disclosure. FIG. 7 is a schematic diagram illustrating an example of a pasting apparatus for forming a barrier layer of the sustained release body according to the embodiment of the disclosure. The method of manufacturing the sustained release body 10 according to the present embodiment includes, for example, a sustained release film forming step, an active ingredient introducing step, a barrier layer forming step, and a cutting step illustrated in FIG. 3.

As illustrated in FIG. 3, in the method of manufacturing the sustained release body 10 (see FIG. 1), first, the sustained release film forming step (step S101) is performed. This sustained release film forming step is a step of forming the sustained release film 2 made of a vapor deposition polymerization film on the front surface 1a (first surface) out of both end surfaces of the substrate 1 in the thickness direction D1. In this sustained release film forming step, for example, a film forming apparatus 30 illustrated in FIG. 5 is used. As illustrated in FIG. 5, the film forming apparatus 30 includes a vacuum tank 31 as a reaction chamber, a connecting unit 32, a housing unit 33, and a vapor deposition polymerization film forming unit 37.

An exhaust pipe (not illustrated) communicating with a vacuum pump (not illustrated) is connected to the vacuum tank 31, and the vacuum tank 31 is communicably joined to the housing unit 33 via the connecting unit 32 having a hollow structure. By operation of the vacuum pump of the vacuum tank 31, air inside the film forming apparatus 30 (specifically, inside the vacuum tank 31, the connecting unit 32, and the housing unit 33) is discharged to the outside through the exhaust pipe, whereby the inside of the film forming apparatus 30 is brought into a vacuum state.

In addition, as illustrated in FIG. 5, a main roller 34 is disposed inside the vacuum tank 31, and an unwinding roller 35 and a winding roller 36 are disposed inside the housing unit 33. Note that each of the main roller 34 and the winding roller 36 is configured to be rotationally driven continuously by a rotary drive device (not illustrated) such as an electric motor.

Furthermore, a roll obtained by winding the belt-shaped substrate 1 to be the substrate 1 of the sustained release body 10 is attached to the unwinding roller 35. The belt-shaped substrate 1 unwound from the unwinding roller 35 is wound on the main roller 34, and the belt-shaped substrate 1 fed from the main roller 34 is wound around the winding roller 36. That is, in the film forming apparatus 30, the belt-shaped substrate 1 is conveyed by a so-called roll-to-roll method from the unwinding roller 35 to the winding roller 36 via the main roller 34. Note that, as illustrated in FIG. 5, a plurality of tension rollers 38a to 38h is disposed on a conveying path in the film forming apparatus 30.

Specifically, in step S101 illustrated in FIG. 3, first, the belt-shaped substrate 1 is unwound from the unwinding roller 35 by rotary drive of the main roller 34 and the winding roller 36. The unwound belt-shaped substrate 1 is conveyed in one circumferential direction (a direction indicated by an arrow a in FIG. 5) on an outer peripheral surface of the main roller 34. While the belt-shaped substrate 1 is being conveyed along the outer peripheral surface of the main roller 34, a vapor deposition polymerization film is formed on the belt-shaped substrate 1 by the vapor deposition polymerization film forming unit 37. The belt-shaped substrate 1 on which the vapor deposition polymerization film is formed in this manner is wound by the winding roller 36.

For example, as illustrated in FIG. 5, the vapor deposition polymerization film forming unit 37 includes two storage pots 39a and 39b and a heater (not illustrated) for heating each of the two storage pots 39a and 39b. In the storage pots 39a and 39b, raw material monomers 40a and 40b of the vapor deposition polymerization film constituting the sustained release film 2 are stored in a liquid state under a vacuum environment, respectively. In addition, the vapor deposition polymerization film forming unit 37 includes vapor supply pipes 41a and 41b, on-off valves 42a and 42b, and a mixing tank 43. The vapor supply pipes 41a and 41b have first end openings connected to the storage pots 39a and 39b and other end openings connected to the mixing tank 43, and communicably connect the storage pots 39a and 39b to the mixing tank 43, respectively. The on-off valves 42a and 42b are disposed at intermediate portions of the vapor supply pipes 41a and 41b, respectively. The mixing tank 43 includes an external tank disposed outside the vacuum tank 31 and an internal tank disposed inside the vacuum tank 31. As illustrated in FIG. 5, the vapor supply pipes 41a and 41b are connected to the external tank of the vacuum tank 31. The internal tank of the vacuum tank 31 is configured to communicate with the external tank, and has a blowout port 44 at an end on the main roller 34 side. The blowout port 44 is disposed inside the vacuum tank 31 so as to face an outer peripheral surface of the main roller 34.

In such a vapor deposition polymerization film forming unit 37, two or more types of raw material monomers (two types of raw material monomers 40a and 40b in FIG. 5) vaporized by the heater are supplied from the storage pots 39a and 39b to the mixing tank 43 through the vapor supply pipes 41a and 41b, respectively, and are sprayed from the blowout port 44 on the front surface 1a of the belt-shaped substrate 1. These two or more types of raw material monomers are sequentially vapor-deposited on the front surface 1a of the belt-shaped substrate 1, and polymerization of these two or more types of raw material monomers proceeds on the front surface 1a. As a result, the sustained release film 2 made of a vapor deposition polymerization film is sequentially formed on the front surface 1a of the belt-shaped substrate 1 while following minute unevenness present on the front surface 1a. As a result, as illustrated in FIG. 4, a belt-shaped body 15 in which the sustained release film 2 is formed on the front surface 1a of the belt-shaped substrate 1 is prepared (state A1). After formation of the sustained release film 2 is completed, the belt-shaped body 15 is conveyed from the main roller 34 to the inside of the housing unit 33 through the inside of the connecting unit 32 as illustrated in FIG. 5. An in-line monitor 45 is disposed inside the housing unit 33, and the belt-shaped body 15 conveyed into the housing unit 33 passes through a measurement region of the in-line monitor 45. At this time, the in-line monitor 45 measures the film thickness and the film quality of the belt-shaped body 15. Thereafter, the belt-shaped body 15 is wound in a roll shape by the winding roller 36, taken out from the housing unit 33 in a state of being wound in a roll shape, and conveyed to the next step.

Examples of the raw material monomer include an amine and an isocyanate. Examples of the amine include tris(2-aminoethyl) amine, diethylenetriamine, dipropylenetriamine, bishexamethylenetriamine, 1,6-diaminohexane, 1,12-diaminododecane, 4,4-diphenyldiaminomethane, 1,4-diaminobenzene, and m-xylylenediamine. Examples of the isocyanate include isophorone diisocyanate, 1,3-bis(isocyanatomethyl) cyclohexane, hexamethylene diisocyanate, dicyclohexylmethane 4,4′-diisocyanate, m-xylylene diisocyanate, and methylene diphenyl 4,4′-diisocyanate.

Examples of the vapor deposition polymerization film constituting the sustained release film 2 include a film of a vapor deposition polymer such as a polyurea resin, a polyamide resin, a polyimide resin, a polyamideimide resin, a polyester resin, a polyazomethine resin, or a polyurethane resin generated by a polymerization reaction between an amine and an isocyanate. Among the vapor deposition polymers described above, the polyurea resin and the polyurethane resin are polymers obtained by a polyaddition reaction, therefore have a feature that no by-product is generated, and thus are suitably used as vapor deposition polymerization films constituting the sustained release film 2. In particular, the polyurea resin is most suitable when used as the sustained release film 2 because the polyurea resin has a high reaction rate, and therefore can shorten a conveyance path length and a conveyance time required for curing after formation of the vapor deposition polymerization film, or does not need an apparatus for accelerating the curing, such as a heater. Note that the sustained release film 2 made of a vapor deposition film may be formed on the front surface 1a of the belt-shaped substrate 1 by vapor-depositing one type of raw material monomer vaporized by heating.

After the sustained release film forming step in step S101 described above is performed, the active ingredient introducing step (step S102) is performed as illustrated in FIG. 3. This active ingredient introducing step is a step of introducing the vaporizable active ingredient 11 into the substrate 1 from the back surface 1b (second surface) opposite to the front surface 1a on which the sustained release film 2 is formed out of both end surfaces of the substrate 1 in the thickness direction D1. In this active ingredient introducing step, for example, an application apparatus 50 illustrated in FIG. 6 is used. As illustrated in FIG. 6, the application apparatus 50 includes an unwinding unit 51, an application unit 52, and a winding unit 53.

As illustrated in FIG. 6, the unwinding unit 51 includes an unwinding roller 54. A roll obtained by winding the belt-shaped body 15 is attached to the unwinding roller 54. As illustrated in FIG. 6, the application unit 52 includes an application chamber 55. The application chamber 55 is a box-shaped structure having an inlet and an outlet through which an object to be subjected to an application treatment passes. Inside the application chamber 55, a storage pot (not illustrated) that stores the active ingredient 11 and a dispenser 57 for introducing the active ingredient 11 into the substrate 1 of the belt-shaped body 15 are disposed. As illustrated in FIG. 6, the winding unit 53 includes a winding roller 58. The winding roller 58 winds the belt-shaped body 15 in a state where the active ingredient 11 is introduced into the substrate 1 in a roll shape.

In addition, the roll-shaped belt-shaped body 15 attached to the unwinding roller 54 passes through the inside of the application chamber 55, is wound on the winding roller 58, and is wound by rotary drive of the winding roller 58. That is, the belt-shaped body 15 is conveyed by a so-called roll-to-roll method from the unwinding roller 54 to the winding roller 58 via the inside of the application chamber 55.

Specifically, in step S102, first, the roll-shaped belt-shaped body 15 wound in step S101 described above is unwound from the unwinding roller 54 and conveyed in a direction toward the application chamber 55 (a direction indicated by an arrow (3 in FIG. 6). Then, the belt-shaped body 15 is carried into the application chamber 55, and is carried to the outside of the application chamber 55 through a discharge region of the dispenser 57. At this time, the belt-shaped body 15 is conveyed such that the back surface 1b of the substrate 1 faces a discharge port side of the dispenser 57. The dispenser 57 sequentially applies a prescribed amount of the active ingredient 11 to the back surface 1b of the substrate 1 of the belt-shaped body 15 conveyed in this manner. Here, as illustrated in FIG. 4, the sustained release film 2 has already been formed on the front surface 1a of the substrate 1 of the belt-shaped body 15. The substrate 1 of the belt-shaped body 15 is impregnated with the active ingredient 11 applied as described above from the back surface 1b opposite to the front surface 1a on which the sustained release film 2 has already been formed (state A2). As a result, a desired amount of the active ingredient 11 is introduced into the substrate 1 of the belt-shaped body 15. After introduction of the active ingredient 11 into the substrate 1 is completed as described above, the belt-shaped body 15 is wound in a roll shape by the winding roller 58, taken out from the application apparatus 50, and conveyed to the next step.

Examples of a method of introducing the active ingredient 11 into the substrate 1 include a wet application method of applying the liquid active ingredient 11. Examples of the wet application method of applying the active ingredient 11 include the dispenser method described above, an inkjet method (inkjet/print printing method), a spin coating method, a dip coating method, a flow coating method, a screen printing method, a die coating method, a wipe coating method, and a gravure roller method.

After the active ingredient introducing step in step S102 described above is performed, the barrier layer forming step (step S103) is performed as illustrated in FIG. 3. This barrier layer forming step is a step of forming the barrier layer 3 that blocks the vaporized gas 11a of the active ingredient 11 on the back surface 1b of the substrate 1 after introduction of the active ingredient 11. In the present embodiment, as an example of the barrier layer forming step, a step in which formation of the barrier layer 3 on the back surface 1b of the substrate 1 and formation of the barrier layer 7 on the front surface 2a of the sustained release film 2 are performed in parallel will be described. In this barrier layer forming step, for example, a pasting apparatus 60 illustrated in FIG. 7 is used. As illustrated in FIG. 7, the pasting apparatus 60 includes an unwinding unit 61, a pasting unit 62, and a winding unit 63.

As illustrated in FIG. 7, the unwinding unit 61 includes an unwinding roller 64. A roll obtained by winding the belt-shaped body 15 after the above-described active ingredient introducing step is attached to the unwinding roller 64. As illustrated in FIG. 7, the pasting unit 62 includes a pair of pressing rollers 65a and 65b, two barrier layer rollers 66a and 66b, and two protective sheet winding rollers 68a and 68b. The pressing rollers 65a and 65b are for pasting barrier layer films 16a and 16b to the substrate 1 and the sustained release film 2 of the belt-shaped body 15, respectively. Specifically, each of outer peripheral surfaces of the pressing rollers 65a and 65b is made of an elastic member. As illustrated in FIG. 7, the pressing rollers 65a and 65b are configured to be rotationally driven while pressing the outer peripheral surfaces of each other. The barrier layer rollers 66a and 66b are for unwinding the barrier layer films 16a and 16b toward a space between the pressing rollers 65a and 65b, respectively. The protective sheet winding rollers 68a and 68b are for winding the protective sheets from the barrier layer films 16a and 16b, respectively. As illustrated in FIG. 7, the winding unit 63 includes a winding roller 67. The winding roller 67 is for winding the belt-shaped body 15 on which the barrier layer films 16a and 16b are pasted in a roll shape.

In addition, the roll-shaped belt-shaped body 15 attached to the unwinding roller 64 passes through a space between the pair of pressing rollers 65a and 65b and is wound on the winding roller 67, and is wound by rotary drive of the pressing rollers 65a and 65b and the winding roller 67. That is, the belt-shaped body 15 is conveyed by a so-called roll-to-roll method from the unwinding roller 64 to the winding roller 67 via a space between the pair of pressing rollers 65a and 65b. Note that, as illustrated in FIG. 7, a plurality of tension rollers 69a to 69c is disposed on a conveying path in the pasting apparatus 60.

Specifically, in step S103, first, the roll-shaped belt-shaped body 15 wound in step S102 described above is unwound from the unwinding roller 64 and conveyed in a direction toward a space between the pair of pressing rollers 65a and 65b (a direction indicated by an arrow y in FIG. 7). Then, the belt-shaped body 15 passes through a space between the pressing rollers 65a and 65b while being sandwiched and pressed by the pair of pressing rollers 65a and 65b. At this time, the barrier layer film 16a unwound from the barrier layer roller 66a and the barrier layer film 16b unwound from the barrier layer roller 66b are pasted on both surfaces of the belt-shaped body 15 in the thickness direction (the front surface 2a of the sustained release film 2 and the back surface 1b of the substrate 1), respectively.

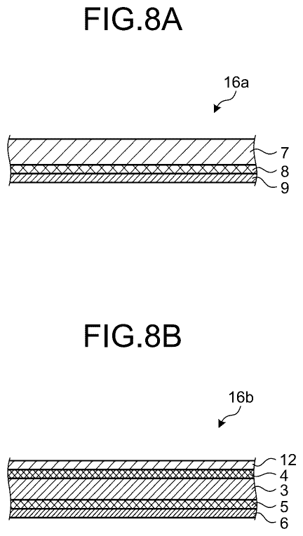

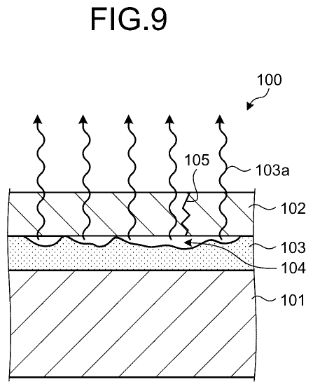

In the present embodiment, the barrier layer film 16a is for forming the barrier layer 7 on the front surface 2a of the sustained release film 2. FIG. 8A is a schematic cross-sectional view illustrating a configuration example of a barrier layer film to be pasted on a sustained release film surface of the sustained release body according to the embodiment of the disclosure. As illustrated in FIG. 8A, the barrier layer film 16a is a belt-shaped laminate having a laminated structure of the barrier layer 7, the adhesive layer 8, and a protective sheet 9. The barrier layer film 16a is attached to the barrier layer roller 66a in a state of being wound in a roll shape. At this time, the barrier layer film 16a is disposed such that the adhesive layer 8 faces the front surface 2a of the sustained release film 2 of the belt-shaped body 15. As illustrated in FIG. 7, before the barrier layer film 16a unwound from the barrier layer roller 66a is pasted on the belt-shaped body 15, only the protective sheet 9 is wound by the protective sheet winding roller 68a, and then the barrier layer film 16a is conveyed to the pressing roller 65a. Then, the barrier layer film 16a from which the protective sheet 9 has been peeled off is pasted on the front surface 2a of the sustained release film 2 of the belt-shaped body 15 by being sandwiched between the pressing rollers 65a and 65b. As a result, the barrier layer 7 is formed on the front surface 2a of the sustained release film 2 via the adhesive layer 8.

The barrier layer film 16b is for forming the barrier layer 3 on the back surface 1b of the substrate 1 containing the active ingredient 11. FIG. 8B is a schematic cross-sectional view illustrating a configuration example of a barrier layer film to be pasted on a substrate surface of the sustained release body according to the embodiment of the disclosure. As illustrated in FIG. 8B, the barrier layer film 16b is a belt-shaped laminate having a laminated structure of a protective sheet 12, the adhesive layer 4, the barrier layer 3, the adhesive layer 5, and the protective sheet 6. The barrier layer film 16b is attached to the barrier layer roller 66b in a state of being wound in a roll shape. At this time, the barrier layer film 16b is disposed such that the adhesive layer 4 faces the back surface 1b of the substrate 1 of the belt-shaped body 15. As illustrated in FIG. 7, before the barrier layer film 16b unwound from the barrier layer roller 66b is pasted on the belt-shaped body 15, only the protective sheet 12 is wound by the protective sheet winding roller 68b, and then the barrier layer film 16b is conveyed to the pressing roller 65b. Then, the barrier layer film 16b from which the protective sheet 12 has been peeled off is pasted on the back surface 1b of the substrate 1 of the belt-shaped body 15 by being sandwiched between the pressing rollers 65a and 65b. As a result, a laminate of the barrier layer 3, the adhesive layer 5, and the protective sheet 6 is formed on the back surface 1b of the substrate 1 via the adhesive layer 4.

When the belt-shaped body 15 unwound from the unwinding roller 64 passes through a space between the pair of pressing rollers 65a and 65b, the adhesive layer 8 of the barrier layer film 16a is pressed against the front surface 2a of the sustained release film 2, and the adhesive layer 4 of the barrier layer film 16b is pressed against the back surface 1b of the substrate 1. Therefore, the belt-shaped body 15 after passing through a space between the pressing rollers 65a and 65b has a laminated structure in which the barrier layer 7, the adhesive layer 8, the sustained release film 2, the substrate 1 containing the active ingredient 11, the adhesive layer 4, the barrier layer 3, the adhesive layer 5, and the protective sheet 6 are laminated in the thickness direction. Thereafter, the belt-shaped body 15 is wound in a roll shape by the winding roller 67, taken out from the pasting apparatus 60 in a state of being wound in a roll shape, and conveyed to the next step.

Note that the treatment of pasting the barrier layer 3 and the treatment of pasting the barrier layer 7 may be performed in parallel as described above in one barrier layer forming step, or a treatment of pasting one of the barrier layers may be performed before a treatment of pasting the other barrier layer. By such a step S103, as illustrated in FIG. 4, the belt-shaped body 15 having a laminated structure of the substrate 1 containing the active ingredient 11, the sustained release film 2 formed on the front surface 1a of the substrate 1, the adhesive layer 4 formed on the back surface 1b of the substrate 1, the barrier layer 3, the adhesive layer 5, the protective sheet 6, the adhesive layer 8 formed on the front surface 2a of the sustained release film 2, and the barrier layer 7 is formed (state A3).

After the barrier layer forming step in step S103 described above is performed, the cutting step (step S104) is performed as illustrated in FIG. 3. This cutting step is a step of cutting the belt-shaped body 15 described above into the target sustained release body 10.

Specifically, in step S104, first, the belt-shaped body 15 after the barrier layer forming step is performed is set in a cutting apparatus in a state of being wound in a roll shape. This cutting apparatus sequentially cuts the belt-shaped body 15 while pulling out the belt-shaped body 15 from the set roll and conveying the belt-shaped body 15. At this time, for example, as illustrated in FIG. 4, the sustained release body 10 having a desired size is sequentially cut out from the sequentially conveyed belt-shaped body 15 (state A4). As described above, the intended sustained release body 10 is sequentially manufactured.

As described above, in the sustained release body 10 according to the embodiment of the disclosure, the sustained release film 2 made of a vapor deposition polymerization film is formed on the front surface 1a out of both end surfaces (front and back surfaces) of the substrate 1 in the thickness direction D1, and the sustained release film 2 is configured to receive the vaporized gas 11a of the vaporizable active ingredient 11 contained in the substrate 1 from the front surface 1a of the substrate 1 and sustainedly release the vaporized gas 11a.

Therefore, the sustained release film 2 is brought into direct close contact with the front surface 1a of the substrate 1 along the minute unevenness of the front surface 1a of the substrate 1, the close contact state between the substrate 1 and the sustained release film 2 is maintained, and the vaporized gas 11a of the active ingredient 11 vaporized from the substrate 1 can be sustainedly released to the outside by the sustained release film 2. This makes it possible to continue sustained release of the vaporized gas 11a of the active ingredient 11 without presence of the active ingredient 11 at a close contact interface between the substrate 1 and the sustained release film 2. Therefore, even if the content of the active ingredient 11 in the substrate 1 decreases with the sustained release of the vaporized gas 11a of the active ingredient 11, it is possible to avoid a situation in which an unintended gap (a gap other than the gas release port on the substrate surface) is generated at the close contact interface between the substrate 1 and the sustained release film 2. As a result, since the sustained release film 2 can be prevented from being damaged by the gap, it is possible to continue sustained release of the vaporized gas 11a of the active ingredient 11 stably over a target long period of time without excessively releasing the vaporized gas 11a of the active ingredient 11 from a damaged portion of the sustained release film 2.

By using the sustained release body 10 as described above, it is possible to cause the active ingredient 11 to act on a desired object over a long period of time, and it is possible to prevent a situation in which fragments of the sustained release film 2 detached from the sustained release body 10 and an excessive amount of the vaporized gas 11a of the active ingredient 11 released from the damaged portion of the sustained release film 2 adhere to the object to damage the object. For example, when an antifungal agent is used as the active ingredient 11 and the antifungal agent vaporized from the sustained release body 10 is caused to act on food such as fruits, it is possible to prevent a situation in which the food is damaged by fragments of the sustained release film 2 or a vaporized gas of the antifungal agent excessively released.

In addition, as a technique for imparting a sustained release function to the substrate surface (active ingredient release surface), it is also conceivable to paste a sustained release film to the substrate surface or to apply a sustained release material to the substrate surface in addition to forming a vapor deposition polymerization film on the substrate surface. However, in a case where a sustained release film manufactured by a method other than vapor deposition polymerization is used, adhesion between the sustained release film and the substrate is significantly poor as compared with the vapor deposition polymerization film, and therefore it is necessary to devise a method such as forming an adhesive layer between the sustained release film and the substrate or bringing the sustained release film into close contact with the substrate surface by heating. As a result, a problem such as a decrease in vaporization ability of the active ingredient occurs. In addition, in a case where a sustained release material is applied to the substrate surface, variation in application thickness occurs, and therefore it is difficult to achieve a uniform sustained release property on the substrate surface. On the other hand, in the disclosure, the minute uneven surface of the substrate that releases the vaporized gas of the active ingredient and the vapor deposition polymerization film having a sustained release property are combined, whereby it is possible to achieve both improvement of adhesion between the substrate surface and the sustained release film and improvement of the sustained release function by the sustained release film, and a crack of the sustained release film is less likely to be generated.

In the method of manufacturing a sustained release body according to the embodiment of the disclosure, the sustained release film forming step of forming the sustained release film 2 made of a vapor deposition polymerization film on the front surface 1a of the substrate 1 is performed, and then the active ingredient introducing step of introducing the vaporizable active ingredient 11 into the substrate 1 from the back surface 1b of the substrate 1 (a surface opposite to the front surface 1a on which the sustained release film 2 is formed) is performed. As a result, it is possible to easily manufacture the sustained release body 10 that exhibits the above-described action and effect.

In addition, since the active ingredient introducing step is performed after the sustained release film forming step to perform these two steps separately, the vaporizable active ingredient does not enter the vacuum chamber (vacuum tank), and as a result, vaporization of the active ingredient under a vacuum state does not occur. Therefore, it is possible to prevent a decrease in yield in manufacture of the sustained release body. In addition, it is possible to prevent the inside of the vacuum chamber from being contaminated by the vaporized active ingredient.

In addition, in the sustained release body 10 and the method of manufacturing the sustained release body 10 according to the embodiment of the disclosure, the barrier layer 3 that blocks the vaporized gas 11a of the active ingredient 11 is formed on the back surface 1b opposite to the front surface 1a (a surface on which the sustained release film 2 is formed) of the substrate 1. Therefore, it is possible to prevent a situation in which the vaporized gas 11a of the active ingredient 11 is unintentionally released from the back surface 1b of the substrate 1, that is, a surface opposite to the front surface 1a from which release (sustained release) of the vaporized gas 11a of the active ingredient 11 is intended. As a result, it is possible to suppress excessive and wasteful release of the vaporized gas 11a of the active ingredient 11 from the sustained release body 10, and it is possible to sustainedly release the vaporized gas 11a of the active ingredient 11 efficiently via the sustained release film 2. As a result, more stable sustained release of the vaporized gas 11a of the active ingredient 11 can be continued over a target long period of time.

### EXAMPLE

Next, the disclosure will be specifically described on the basis of Example. In Example, in order to confirm the effect of the disclosure, the following experiments were performed. Note that the disclosure is not limited to the following Example.

First, in Example, a film obtained by adding calcium carbonate to a polyethylene film having a size of length: 10 cm×width: 10 cm×thickness: 75 μm and stretching the film to make the film porous (porous film) was adopted as a substrate. Next, a sustained release film made of a vapor deposition polymerization film was formed on a first surface (front surface) out of both end surfaces of the porous film in the thickness direction by a known vapor deposition polymerization method using two types of raw material monomers. In Example, diethylenetriamine and 1,3-bis(isocyanatomethyl) cyclohexane were used as the two types of raw material monomers. In this case, the vapor deposition polymerization film constituting the sustained release film of Example is a polyurea resin. Thereafter, a vaporizable active ingredient was introduced into a second surface (back surface) opposite to the first surface (front surface) out of both end surfaces of the porous film in the thickness direction by a dispenser method. In Example, hinokitiol was used as the active ingredient. A sample of Example was prepared as described above.

Meanwhile, in Comparative Example of the disclosure, a polyethylene film having a solid structure and hardly impregnated with the active ingredient was used as the substrate. Note that the size of the substrate in Comparative Example is the same as that in the above-described Example. Next, a vaporizable active ingredient was applied to a first surface out of both end surfaces of the polyethylene film in the thickness direction by a dispenser method, thereby forming an active ingredient layer on the first surface. The active ingredient in Comparative Example is the same as that in the above-described Example. Thereafter, on a surface of the active ingredient layer, a sustained release film made of a vapor deposition polymerization film was formed by a known vapor deposition polymerization method using two types of raw material monomers. The two types of raw material monomers and vapor deposition polymerization film in Comparative Example are the same as those in the above-described Example. A sample of Comparative Example was prepared as described above.

For each of the samples of Example and Comparative Example prepared as described above, an experiment of leaving the samples in a thermostatic bath was performed, and presence or absence of generation of cracks in the sustained release film in each of the samples was thereby evaluated. In this experiment, 20° C. and 30° C. were used as a temperature condition in the thermostatic bath, and normal pressure was used as a pressure condition in the thermostatic bath. In addition, for each of the samples of Example and Comparative Example, presence or absence of generation of cracks in the sustained release film was visually evaluated by a plurality of operators immediately after production and after 48 hours, 168 hours, one month, and three months had elapsed from start of leaving the samples in the thermostatic bath. As a criterion for this visual evaluation, the size of a target crack was defined to be 2 mm or more, and the number of cracks generated per cm² in the sustained release film of each of the samples was counted. Evaluation results of Example and Comparative Example are illustrated in Table 1. In Table 1, “◯” means that no crack is generated in the sustained release film (the number of cracks generated=0), and “x” means that a crack is generated in the sustained release film (the number of cracks generated ≥1).

TABLE 1

| | Temperature | | After | After | After | After |
|---|---|---|---|---|---|---|
| | of | Immediately | elapse of | elapse of | elapse of | elapse of |
| | thermostatic | after | 48 | 168 | one | three |
| | bath | production | hours | hours | month | months |
| Example | 20° C. | ◯ | ◯ | ◯ | ◯ | ◯ |
| | 30° C. | ◯ | ◯ | ◯ | ◯ | ◯ |
| Comparative | 20° C. | ◯ | ◯ | ◯ | X | X |
| Example | 30° C. | ◯ | ◯ | X | X | X |

As is obvious with reference to Table 1, in the sample of Comparative Example, a crack of the sustained release film was generated after one month had elapsed under the temperature condition of 20° C., and a crack of the sustained release film was generated after 168 hours had elapsed under the temperature condition of 30° C. On the other hand, in the samples of Example, no crack of the sustained release film was generated regardless of the temperature condition of the thermostatic bath even after three months had elapsed from start of leaving the sample. From this evaluation result, it has been confirmed that according to the sustained release body of the disclosure, a stable sustained release amount of the vaporized active ingredient can be continued over a long period of time.

Note that, in the above-described embodiment, the case where the liquid active ingredient 11 is contained in the substrate 1 has been exemplified, but the disclosure is not limited thereto. For example, the solid active ingredient 11 may be contained in the substrate 1, for example, by embedding the solid active ingredient 11 in the substrate 1, and the vaporized gas 11a sublimated from the solid active ingredient 11 may be sustainedly released.

In addition, in the above-described embodiment, the barrier layer 3 that blocks the vaporized gas 11a of the active ingredient 11 is formed on the back surface 1b of the substrate 1, but the disclosure is not limited thereto. For example, the barrier layer 3 may be formed not only on the back surface 1b of the substrate 1 but also on the side surface 1c of the substrate 1. That is, the barrier layer 3 may be configured to cover the back surface 1b and the side surface 1c of the substrate 1 (remaining substrate surfaces excluding the front surface 1a on which the sustained release film 2 is formed). Alternatively, the sustained release film 2 may be formed not only on the front surface 1a of the substrate 1 but also on the side surface 1c of the substrate 1. That is, the sustained release film 2 may be configured to cover the front surface 1a and the side surface 1c of the substrate 1 (remaining substrate surfaces excluding the back surface 1b on which the barrier layer 3 is formed). The vaporized gas 11a of the active ingredient 11 may be thereby sustainedly released from the front surface 1a and the side surface 1c of the substrate 1 via the sustained release film 2.

In addition, in the above-described embodiment, the barrier layer 3 on the back surface 1b of the substrate 1 and the barrier layer 7 on the front surface 2a of the sustained release film 2 are formed in one step (in the barrier layer forming step in step S103 illustrated in FIG. 3), but the disclosure is not limited thereto. For example, these barrier layers 3 and 7 may be formed on the substrate 1 or the sustained release film 2 in separate steps. At this time, a step of forming the barrier layer 3 on the back surface 1b of the substrate 1 may be performed first, and then a step of forming the barrier layer 7 on the front surface 2a of the sustained release film 2 may be performed, or vice versa.

In addition, in the above-described embodiment, the case has been exemplified where a laminate (for example, the barrier layer film 16b) having a laminated structure of the barrier layer 3, the adhesive layer 5, and the like is formed on the back surface 1b of the substrate 1 from the barrier layer 3 side via the adhesive layer 4, but the disclosure is not limited thereto. For example, the barrier layer 3 and the adhesive layer 5 with the protective sheet 6 may be configured separately from each other, the barrier layer 3 may be formed on the back surface 1b of the substrate 1, and then the adhesive layer 5 and the protective sheet 6 may be sequentially formed on the back surface of the barrier layer 3.

In addition, the disclosure is not limited by the sustained release body and the method of manufacturing the sustained release body according to the above-described embodiment, and the disclosure also includes those configured by appropriately combining the above-described components and manufacturing steps. For example, the active ingredient introducing step in step S102 and the barrier layer forming step in step S103 may be performed with one apparatus, or the active ingredient introducing step in step S102, the barrier layer forming step in step S103, and the cutting step in step S104 may be performed with one apparatus. In addition, other embodiments, Example, operation techniques, and the like performed by a person skilled in the art or the like on the basis of the above-described embodiment are all included in the scope of the disclosure.

The disclosure exhibits an effect that stable sustained release of an active ingredient can be continued over a target long period of time.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A sustained release body comprising:
a substrate containing a vaporizable active ingredient, the substrate having a structure for releasing a vaporized gas of the active ingredient; and
a vapor deposition polymerization film directly formed on a first surface that is one end surface of both end surfaces of the substrate in a thickness direction of the substrate, the vapor deposition polymerization film being configured to receive the vaporized gas of the active ingredient from the first surface of the substrate and sustainedly release the vaporized gas.

2. The sustained release body according to claim 1, comprising
a barrier layer formed on a second surface opposite to the first surface, the second surface being another end surface of the both end surfaces of the substrate in the thickness direction, the barrier layer being configured to block the vaporized gas of the active ingredient from the second surface of the substrate.

3. The sustained release body according to claim 1, wherein
the substrate is made of a porous material.

4. The sustained release body according to claim 3, wherein
the porous material has a structure of continuous open cell.

5. The sustained release body according to claim 1, wherein
the substrate is made of a polymer having a non-crystalline portion.

6. The sustained release body according to claim 1, wherein
the vapor deposition polymerization film has lower gas permeability than the substrate.

7. The sustained release body according to claim 1, wherein
the vapor deposition polymerization film has a thickness in a range of 1 μm or more and 900 μm or less.

8. A method of manufacturing a sustained release body, comprising:
forming a sustained release film made of a vapor deposition polymerization film on a first surface that is one end surface of both end surfaces of the substrate in a thickness direction of the substrate; and
introducing a vaporizable active ingredient into the substrate from a second surface opposite to the first surface, the second surface being another surface of the both end surfaces of the substrate in the thickness direction.

9. The method of manufacturing a sustained release body according to claim 8, comprising
forming a barrier layer configured to block a vaporized gas of the active ingredient on the second surface of the substrate after introduction of the active ingredient.
